(19) United States
(12) Patent Application Publication
(10) Pub. No.: US 2022/0370738 A1
(43) Pub. Date: Nov. 24, 2022

(54) FILLING HEAD FOR POWERED LAYERING OF A DRY POWDER INHALER

(71) Applicants: MYLAN PHARMA UK LIMITED, Hertfordshire (GB); PFIZER LIMITED, Kent (GB)

(72) Inventors: Simon Cooper, Hertfordshire (GB); Bruce MacMichael, Kent (GB); Peter Houzego, Kent (GB)

(73) Assignees: MILAN PHARMA UK LIMITED, Hertfordshire (GB); PFIZER LIMITED, Kent (GB)

(21) Appl. No.: 17/621,602

(22) PCT Filed: Jun. 24, 2020
(86) PCT No.: PCT/IB2020/055995

§ 371 Date: Dec. 21, 2021

Related U.S. Application Data
(60) Provisional application No. 62/866,167, filed on Jun. 25, 2019.

(51) Int. Cl. A61M 15/00

(52) U.S. Cl. CPC A61M 15/0048; A61M 2209/045

(57) ABSTRACT

A filling head (20) for filling a container (22) includes a receptacle (24). The receptacle (24) includes an interior surface (26) configured to receive a powder (28) and having a plurality of openings (34) disposed therein to allow passage of the powder (28); and an exterior surface (52). The exterior surface (52) of the receptacle (24) has a plurality of raised surfaces (54), each of the plurality of raised surfaces having a plurality of openings (56) aligned with at least one of the plurality of openings (34) of the interior surface (26) to allow passage of the powder (28) from the receptacle (24) to the container (22). Methods of filling a container (22) are also provided.

### CROSS REFERENCE TO RELATED APPLICATION(S)

The present application claims priority to U.S. Provisional Patent Application No. 62/866,167, filed on Jun. 25, 2019, which is incorporated herein by reference in its entirety.

### BACKGROUND

### Field

The present invention relates to a filling head for filling a container and, in particular, a filling head for a unit dose carrier.

### Description of Related Art

Development of inhalation technology for drug delivery has contributed immensely in treating various intrapulmonary and extrapulmonary diseases. This is supported by the lungs' unique geometry such as a large surface area, thin alveolar epithelial lining, high vascularization, and avoidance of first-pass metabolism. Numerous inhalation delivery systems have been developed and studied to treat lung diseases such as asthma, chronic obstructive pulmonary disease (COPD), and other pulmonary infections. Among them, three approaches, that is, nebulizers, pressurized metered-dose inhalers (pMDIs), and dry powder inhalers (DPI), are extensively scrutinized for the treatment of several lung diseases and pathological conditions. Utilization of nebulizers requires bulky compressors or a source of compressed air, while pMDIs have limitations such as sedimentation, crystal growth, and selection of appropriate propellant, and they emit dosages at high velocity, which build deposition in the oropharynx most commonly where they are swallowed and enhance the risk of systemic absorption. DPIs were introduced to cure some of the weaknesses associated with nebulizers and pMDIs.

A DPI is a breath-activated device that aerosolizes a set dose of a micronized drug (e.g., active pharmaceutical ingredient (API)) in the solid state on an airstream. The DPI provides certain advantages regarding drug stability, ease of handling, and range of possible dose weights to be administered. For example, DPIs provide physicochemical stability and deep lungs deposition using the patient's respiration. Additionally, they do not require cold chain storage or reconstitution of powders into solutions for nebulization.

There are different types of DPIs that are available for patient use in different types of containers including bulk powder dispensers, single-dose dispensers, and multiple-dose dispensers. Bulk powder dispensers hold enough bulk powder for a plurality of doses and are typically used with an inhaler having a shuttle or rotary mechanism that transfers a metered dose from the bulk powder reserve into an inhalation chamber in the inhaler. Single-dose dispensers supply a dose of a drug in individual capsules. Before each administration, the patient has to load the device with one capsule for a single dose delivery. Single-dose DPIs can further be classified as disposable or reusable. The third type of DPI, the multiple-dose inhaler, has a number of unit doses secured in individual chambers or pockets. Typically, the packaging includes replaceable disks, cartridges, or strips of foil-polymer blister packaging. Multi-dose inhalers store the powder in bulk and have a built-in mechanism to meter individual doses upon actuation.

The unit doses of a multiple-dose inhaler are conventionally filled with a powdered drug using manual, semi-automated, or automated filling machines. However, depending on the type of machine, significant to slight fill weight variability can occur, as well as powder compression or inconsistent powder compression. Further, when a low fill weight of a single drug or a low fill weight layer of a drug(s) is desired, standard filling machines may not accurately load the unit doses and may not provide layering capabilities for multiple drugs. Additionally, when multiple layers of a drug(s) and/or an excipient(s) are desired to be filled in unit doses, standard filling heads of filling machines may drag a percentage of the powder out of the unit dose after filling takes place.

### SUMMARY

According to an example of the present disclosure, a filling head is provided that is compatible with a machine that produces low fill weight variability, is capable of creating multi-layers of a drug or drugs, and can deliver a small volume of a drug to each of the unit doses. According to the example, the filling head ensures that each dose of a drug is metered into a defined volume at a consistent density, thus achieving desired weight control and uniformity of the drug.

According to an example of the present disclosure, a filling head for filling a container, such as a unit dose carrier, is provided. The filling head enables the container to be filled with a defined amount of powder and also allows the powder to be layered in a linear or non-linear configuration.

According to a particular example of the present disclosure, a filling head for filling a container is provided. The filling head comprises a receptacle. The receptacle comprises an interior surface configured to receive a powder and having a plurality of openings disposed therein to allow passage of the powder; and an exterior surface, the exterior surface of the receptacle having a plurality of raised surfaces, each of the plurality of raised surfaces having a plurality of openings aligned with at least one of the plurality of openings of the interior surface to allow passage of the powder from the receptacle to the container.

The plurality of openings may be circumferentially arrayed about the interior surface, and the plurality of openings of the raised surfaces are circumferentially arrayed about the exterior surface.

The container may comprise a pocket sized to receive at least one of the plurality of raised surfaces of the exterior surface of the receptacle, and the exterior surface of the filling head may comprise a protuberance disposed on an edge of the filling head configured for engagement with an indent on the container. A plurality of pockets may be disposed radially on a disc, each pocket sized to receive at least one of the plurality of raised surfaces of the exterior surface of the receptacle to allow filling of the pocket with the powder.

The powder may comprise a medicament.

The medicament may comprise an excipient.

The powder may comprise a powder for a dry powder inhaler.

The plurality of raised surfaces may have a linear configuration.

The plurality of raised surfaces may have a non-linear or angled configuration.

Each of the plurality of raised surfaces may comprise at least three openings to allow passage of the powder from the receptacle to the container.

A conical-shaped projection may be disposed substantially in a center of the interior surface, the conical-shaped projection defining an interior channel.

The filling head may define a channel disposed substantially in the center of the filling head, the channel being configured for disposal of a fixation element comprising a screw to lock the conical-shaped projection onto the interior surface.

The exterior surface may comprise a first flange and a second flange, the first flange being located opposite the second flange.

According to a particular example of the present disclosure, a filling head for filling a container is provided. The filling head comprises a receptacle. The receptacle comprises an interior surface configured to receive a powder and having a plurality of circumferentially arrayed openings disposed therein to allow passage of the powder; and an exterior surface, the exterior surface of the receptacle having a plurality of circumferentially arrayed raised surfaces, each of the plurality of raised surfaces having a plurality of openings aligned with at least one of the plurality of openings of the interior surface to allow passage of the powder from the receptacle to the container.

The container may comprise a pocket sized to receive at least one of the plurality of raised surfaces of the exterior surface of the receptacle, and a plurality of pockets being disposed radially on a disc, each pocket sized to receive at least one of the plurality of raised surfaces of the exterior surface of the receptacle to allow filling of the pocket with the powder.

The powder may comprise a medicament, the powder may comprise a medicament comprising an excipient, and/or the powder may comprise a powder for a dry powder inhaler.

The plurality of raised surfaces may have a linear configuration, or the plurality of raised surfaces may have a non-linear or angled configuration.

The interior surface may further comprise a conical-shaped projection disposed substantially in a center of the interior surface, and each of the plurality of raised surfaces may comprise at least three openings to allow passage of the powder from the receptacle to the container.

According to a particular example of the present disclosure, a method of filling a container is provided. The method comprises providing a filling head containing a powder, the filling head comprising a receptacle, the receptacle having an interior surface configured to receive the powder and having a plurality of openings disposed therein to allow passage of the powder; the receptacle having an exterior surface, the exterior surface of the receptacle having a plurality of raised surfaces, each of the plurality of raised surfaces having a plurality of openings aligned with at least one of the plurality of openings of the interior surface to allow passage of the powder from the receptacle to the container; and contacting the exterior surface of the filling head with the container to allow passage of the powder from the receptacle into the container.

The powder may comprise a medicament.

The medicament may comprise an excipient.

The powder may comprise a powder for a dry powder inhaler.

According to a particular example of the present disclosure, a method of filling a unit dose carrier using a filling head. The method comprises attaching the filling head to a filling machine, the filling head comprising a receptacle, the receptacle having an interior surface configured to receive a powder and having a plurality of circumferentially arrayed openings disposed therein to allow passage of the powder; the receptacle having an exterior surface, the exterior surface of the receptacle having a plurality of circumferentially arrayed raised surfaces, each of the plurality of raised surfaces having a plurality of openings aligned with at least one of the plurality of openings of the interior surface; loading the interior surface with the powder; engaging the plurality of circumferentially arrayed perforated shapes with a plurality of pockets disposed in the unit dose carrier; attaching the unit dose carrier to the filling head; administering a plurality of taps to the filling head; and filling each of the plurality of the pockets with at least a first layer of powder.

After the interior surface is loaded, the powder may flow through the plurality of circumferentially arrayed openings, out of the plurality of openings of the plurality of circumferentially arrayed raised surfaces, and into each of the plurality of pockets of the unit dose carrier.

The plurality of circumferentially arrayed raised surfaces may engage the plurality of pockets to dispose a defined volume amount of powder into the plurality of pockets. The filling head may dispose at least a first layer, a second layer, a third layer, a fourth layer, and/or a fifth layer of the powder into each of the plurality of pockets. The filling head may fill each of the plurality of pockets with the powder in a total amount from about 2 μ [symbol] to about 32 μ [symbol] .

Further preferred and non-limiting embodiment or aspects will now be described in the following numbered clauses.

Clause 1: A filling head for filling a container, the filling head comprising: a receptacle, the receptacle comprising: an interior surface configured to receive a powder and having a plurality of openings disposed therein to allow passage of the powder; and an exterior surface, the exterior surface of the receptacle having a plurality of raised surfaces, each of the plurality of raised surfaces having a plurality of openings aligned with at least one of the plurality of openings of the interior surface to allow passage of the powder from the receptacle to the container.

Clause 2: The filling head according to clause 1, wherein the plurality of openings are circumferentially arrayed about the interior surface and the plurality of openings of the raised surfaces are circumferentially arrayed about the exterior surface.

Clause 3: The filling head according to clause 1 or clause 2, wherein the container comprises a pocket sized to receive at least one of the plurality of raised surfaces of the exterior surface of the receptacle, and the exterior surface of the filling head comprises a protuberance disposed on an edge of the filling head configured for engagement with an indent on the container.

Clause 4: The filling head according to clause 3, wherein a plurality of pockets are disposed radially on a disc, each pocket sized to receive at least one of the plurality of raised surfaces of the exterior surface of the receptacle to allow filling of the pocket with the powder.

Clause 5: The filling head according to any one of clauses 1-4, wherein the powder comprises a medicament.

Clause 6: The filling head according to clause 5, wherein the medicament comprises an excipient.

Clause 7: The filling head according to any one of clauses 1-6, wherein the powder comprises a powder for a dry powder inhaler.

Clause 8: The filling head according to any one of clauses 1-7, wherein the plurality of raised surfaces have a linear configuration.

Clause 9: The filling head according to any one of clauses 1-7, wherein the plurality of raised surfaces have a non-linear or angled configuration.

Clause 10: The filling head according to any one of clauses 1-9, wherein each of the plurality of raised surfaces comprises at least three openings to allow passage of the powder from the receptacle to the container.

Clause 11: The filling head according to any one of clauses 1-10, wherein a conical-shaped projection is disposed substantially in a center of the interior surface, the conical-shaped projection defining an interior channel.

Clause 12: The filling head according to clause 11, wherein the filling head defines a channel disposed substantially in the center of the filling head, the channel being configured for disposal of a fixation element comprising a screw to lock the conical-shaped projection onto the interior surface.

Clause 13: The filling head according to clause 11 or clause 12, wherein the exterior surface comprises a first flange and a second flange, the first flange being located opposite the second flange.

Clause 14: A filling head for filling a container, the filling head comprising: a receptacle, the receptacle comprising: an interior surface configured to receive a powder and having a plurality of circumferentially arrayed openings disposed therein to allow passage of the powder; and an exterior surface, the exterior surface of the receptacle having a plurality of circumferentially arrayed raised surfaces, each of the plurality of raised surfaces having a plurality of openings aligned with at least one of the plurality of openings of the interior surface to allow passage of the powder from the receptacle to the container.

Clause 15: The filling head according to clause 14, wherein the container comprises a pocket sized to receive at least one of the plurality of raised surfaces of the exterior surface of the receptacle, and a plurality of pockets being disposed radially on a disc, each pocket sized to receive at least one of the plurality of raised surfaces of the exterior surface of the receptacle to allow filling of the pocket with the powder.

Clause 16: The filling head according to clause 14 or clause 15, wherein (i) the powder comprises a medicament, (ii) the powder comprises a medicament comprising an excipient, and/or (iii) the powder comprises a powder for a dry powder inhaler.

Clause 17: The filling head according to any one of clauses 14-16, wherein (i) the plurality of raised surfaces have a linear configuration; and/or (ii) the plurality of raised surfaces have a non-linear or angled configuration.

Clause 18: The filling head according to any one of clauses 14-17, wherein the interior surface further comprises a conical-shaped projection disposed substantially in a center of the interior surface, and each of the plurality of raised surfaces comprising at least three openings to allow passage of the powder from the receptacle to the container.

Clause 19: A method of filling a container, the method comprising: providing a filling head containing a powder, the filling head comprising a receptacle, the receptacle having an interior surface configured to receive the powder and having a plurality of openings disposed therein to allow passage of the powder; the receptacle having an exterior surface, the exterior surface of the receptacle having a plurality of raised surfaces, each of the plurality of raised surfaces having a plurality of openings aligned with at least one of the plurality of openings of the interior surface to allow passage of the powder from the receptacle to the container; and contacting the exterior surface of the filling head with the container to allow passage of the powder from the receptacle into the container.

Clause 20: The method according to clause 19, wherein the powder comprises a medicament.

Clause 21: The method according to clause 20, wherein the medicament comprises an excipient.

Clause 22: The method according to any one of clauses 19-21, wherein the powder comprises a powder for a dry powder inhaler.

Clause 23: A method of filling a unit dose carrier using a filling head, the method comprising: attaching the filling head to a filling machine, the filling head comprising a receptacle, the receptacle having an interior surface configured to receive a powder and having a plurality of circumferentially arrayed openings disposed therein to allow passage of the powder; the receptacle having an exterior surface, the exterior surface of the receptacle having a plurality of circumferentially arrayed raised surfaces, each of the plurality of raised surfaces having a plurality of openings aligned with at least one of the plurality of openings of the interior surface; loading the interior surface with the powder; engaging the plurality of circumferentially arrayed perforated shapes with a plurality of pockets disposed in the unit dose carrier; attaching the unit dose carrier to the filling head; administering a plurality of taps to the filling head; and filling each of the plurality of the pockets with at least a first layer of powder.

Clause 24: The method according to clause 23, wherein after the interior surface is loaded, the powder flows through the plurality of circumferentially arrayed openings, out of the plurality of openings of the plurality of circumferentially arrayed raised surfaces, and into each of the plurality of pockets of the unit dose carrier.

Clause 25: The method according to clause 23 or clause 24, wherein (i) the plurality of circumferentially arrayed raised surfaces engage the plurality of pockets to dispose a defined volume amount of powder into the plurality of pockets; (ii) the filling head disposes at least a first layer, a second layer, a third layer, a fourth layer, and/or a fifth layer of the powder into each of the plurality of pockets; or (iii) the filling head fdls each of the plurality of pockets with the powder in a total amount from about 2 μ [symbol] to about 32 μ [symbol] .

These and other features and characteristics of the present invention, as well as the methods of operation and functions of the related elements of structures and the combination of parts and economies of manufacture, will become more apparent upon consideration of the following description and with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular forms of “a”, “an”, and “the” include plural referents unless the context clearly dictates otherwise.

### BRIEF DESCRIPTION OF THE DRAWINGS

FIG. 1 is a perspective view of a filling head for a container, such as a unit dose carrier, showing an exterior surface thereof, according to an example of the present disclosure;

FIG. 2 is a bottom view of the exterior surface of the filling head of FIG. 1;

FIG. 3 is a perspective view of the filling head of FIG. 1, showing an interior surface thereof;

FIG. 4 is a top view of the interior surface of the filling head of FIG. 1;

FIG. 5 is a partially exploded perspective view of the filling head of FIG. 1, showing the exterior surface thereof;

FIG. 6 is a partially exploded perspective view of the filling head of FIG. 1, showing the interior surface thereof;

FIG. 7 is a top view of a component of the interior surface of the filling head of FIG. 1;

FIG. 8 is an exploded perspective view of components of the filling head of FIG. 1;

FIG. 9 is a side cross-sectional view of the filling head of FIG. 1 contacting a container (e.g., pockets for a dry powder inhaler);

FIG. 9A is an enlarged side cross-sectional view taken from area “FIG. 9A” shown in FIG. 9;

FIG. 10 is an enlarged perspective view of a plurality of raised surfaces disposed on the exterior surface of the filling head of FIG. 1, according to an example of the present disclosure;

FIGS. 10A to 10E are enlarged perspective views of examples of the plurality of raised surfaces shown in FIG. 10;

FIG. 11 is a top view of a filling head for filling a container, such as a unit dose carrier, showing an interior surface thereof, according to an example of the present disclosure;

FIG. 12 is a top view of components of the interior surface of the filling head of FIG. 11;

FIG. 13 is an exploded perspective view of the filling head of FIG. 11, showing an exterior surface thereof;

FIG. 14 is a perspective view and cross-sectional view of a filling machine used with the filling head of FIG. 1, according to an example of the present disclosure;

FIG. 15 is a perspective view of a container in disc form that has individual pockets that are filled with powder by the filling head, according to an example of the present disclosure;

FIG. 16 is a cross-sectional view of various powder layering examples that are inside a pocket of a container produced by the filling head of FIG. 1 and/or FIG. 11;

FIG. 17 is a flow diagram and side view of containers (e.g., discs) being filled with the filling head of FIG. 1;

FIG. 18 is a chart of the results of a comparison between different plurality of raised surfaces shapes; and

FIG. 19 is a side view of an assembled dry powder inhaler having a container, according to an example of the present disclosure.

### DETAILED DESCRIPTION

For purposes of the description hereinafter, the terms “end”, “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings and described in the following specification are simply exemplary embodiments or aspects of the invention. Hence, specific dimensions and other physical characteristics related to the embodiments or aspects disclosed herein are not to be considered as limiting.

Notwithstanding that the numerical ranges and parameters setting forth the broad scope of the application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10; that is, any and all subranges having a minimum value of equal to or greater than 1 and a maximum value of equal to or less than 10, e.g., 5.5 to 10.

### Definitions

Ranges may be expressed herein as from “about” or “approximately” one particular value and/or to “about” or “approximately” another particular value. When such a range is expressed, another embodiment includes from the one particular value and/or to the other particular value.

Spatially relative terms such as “under”, “below”, “lower”, “over”, “upper”, and the like are used for ease of description to explain the positioning of one element relative to a second element. These terms are intended to encompass different orientations of the device in addition to different orientations than those depicted in the figures. Further, terms such as “first”, “second”, or the like are also used to describe various elements, regions, sections, etc. and are also not intended to be limiting. Like terms refer to like elements throughout the description.

As used herein, the terms “having”, “containing”, “including”, “comprising” and the like are open-ended terms that indicate the presence of stated elements or features but do not preclude additional elements or features.

The term “pharmaceutically acceptable salt” or “salt” comprises inorganic and organic salts. Examples of organic salts may include formate, acetate, trifluoroacetate, propionate, butyrate, lactate, citrate, tartrate, malate, maleate, succinate, methanesulfonate, benzenesulfonate, xinafoate, pamoate, and benzoate. Examples of inorganic salts may include fluoride, chloride, bromide, iodide, phosphate, nitrate, and sulphate.

The term “medicament” includes a substance suitable for oral or nasal inhalation. The medicament can include an active pharmaceutical ingredient and an excipient.

As used herein, the term “active pharmaceutical ingredient” (API) includes any substance (i.e., compound or composition of matter) which, when administered to an organism (human or animal), induces a desired pharmacologic and/or physiologic effect by local and/or systemic action. The term therefore encompasses substances traditionally regarded as actives, drugs, or bioactive agents, as well as biopharmaceuticals (e.g., peptides, hormones, nucleic acids, gene constructs, etc.) typically employed to treat a number of conditions which are defined broadly to encompass diseases, disorders, infections, or the like. Exemplary APIs include, without limitation, antibiotics, antivirals, H2-receptor antagonists, 5HT1 agonists, 5HT3 antagonists, COX2-inhibitors, steroids (e.g., prednisone, prednisolone, dexamethasone), APIs used in treating psychiatric conditions such as depression, anxiety, bipolar disorder, tranquilizers, APIs used in treating metabolic conditions, anticancer APIs, APIs used in treating neurological conditions such as epilepsy and Parkinson's Disease, respiratory conditions, such as for example, asthma, Chronic Obstructive Pulmonary Disease (COPD), bronchitis, bronchiolitis, emphysema, or the like. Also included are APIs used in treating cardiovascular conditions, non-steroidal anti-inflammatory APIs, APIs used in treating Central Nervous System conditions, or APIs employed in treating hepatitis. The APIs can be muscarinic M3 receptor agonists or anticholinergic agents, β2-adrenoceptor agonists, compounds having a dual muscarinic antagonist, and P2-agonist activity and glucocorticoid receptor agonists.

In some examples, the API is ipratropium, tiotropium, oxitropium, trospium, aclidiniums, perenzepine, telenzepine, ephedrine, adrenaline, isoprenaline, metaproterenol, phenylephrine, phenylpropanolamine, pirbuterol, reproterol, rimiterol, isoetharine, carmoterol, albuterol, terbutaline, bambuterol, fenoterol, salbutamol, tulobuterol formoterol, salmeterol, prednisone, prednisolone, flunisolide, triamcinolone acetonide, beclomethasone, budesonide, fluticasone, ciclesonide, or mometasone, as well as salts and/or solvates thereof. In some examples, the API is glycopyrronium bromide, formoterol fumarate, and/or tiotropium bromide. In some examples, the API is a glucocorticosteroid, such as, for example, fluticasone, budesonide, mometasone, or ciclesonide. In some examples, the active pharmaceutical ingredient is 5-[3-(3-Hydroxyphenoxy)azetidin-1-yl]-5-methyl-2,2-diphenylhexanamide hydrochloride, which is a long-acting anticholinergic. In some examples, the active pharmaceutical ingredient is 5-[3-(3-Hydroxyphenoxy)azetidin-1-yl]-5-methyl-2,2-diphenylhexanamide, which is a long-acting anticholinergic free base.

The term “excipient” is used to describe an ingredient other than the active pharmaceutical ingredients. The selection of an excipient can depend on factors such as the particular mode of administration, the effect of the excipient on solubility and stability, and the nature of the dosage form.

The term “dry powder inhalers” (DPI) refers to a device that delivers medication to the lungs in the form of a dry powder. DPIs are commonly used to treat respiratory diseases such as asthma, bronchitis, emphysema, and COPD although DPIs. DPIs can be divided into three basic types: i) bulk powder dispensers which hold enough bulk powder for a plurality of doses; ii) single-dose dispensers, for the administration of a single dose of the active compound; and iii) multiple-dose inhalers pre-loaded with quantities of active principles sufficient for longer treatment cycles.

The term “container” refers to a receptacle for a medicament. In some examples, the container can have one or more pockets for the medicament.

The term “unit dose”, “unit dose receptacle”, and/or “dose unit” refers to a container comprising a pocket(s) that contains a medicament configured to be dispensed to a patient at a particular dose.

The headings below are not meant to limit the disclosure in any way; embodiments under any one heading may be used in conjunction with embodiments under any other heading.

### Filling Head

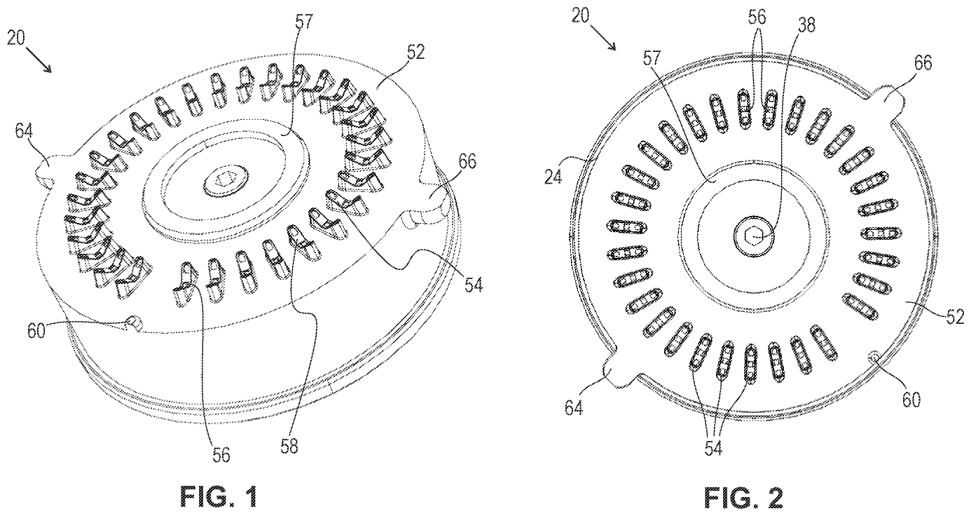

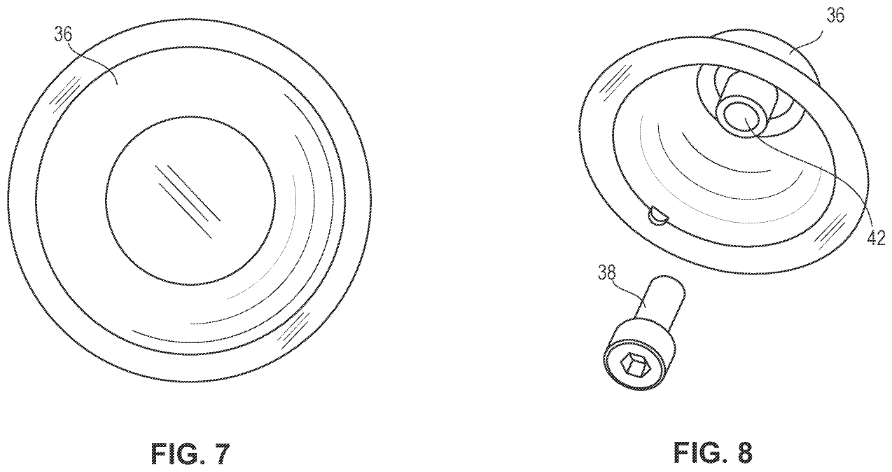

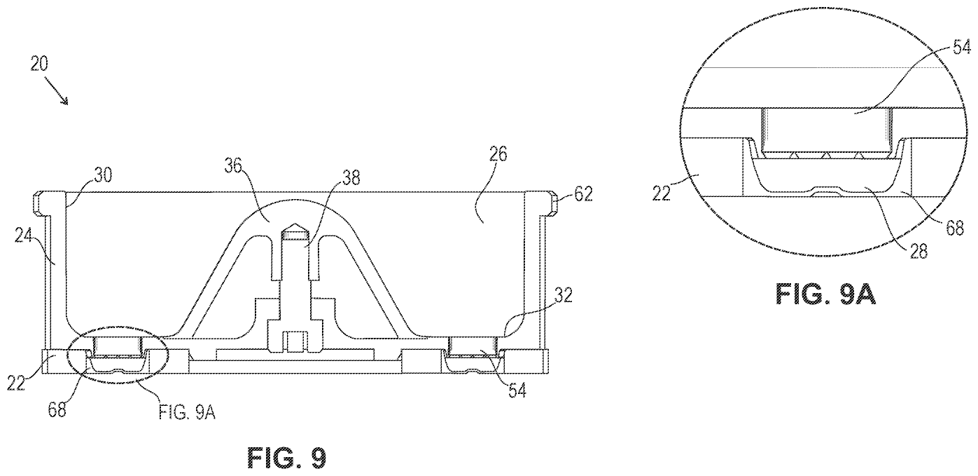

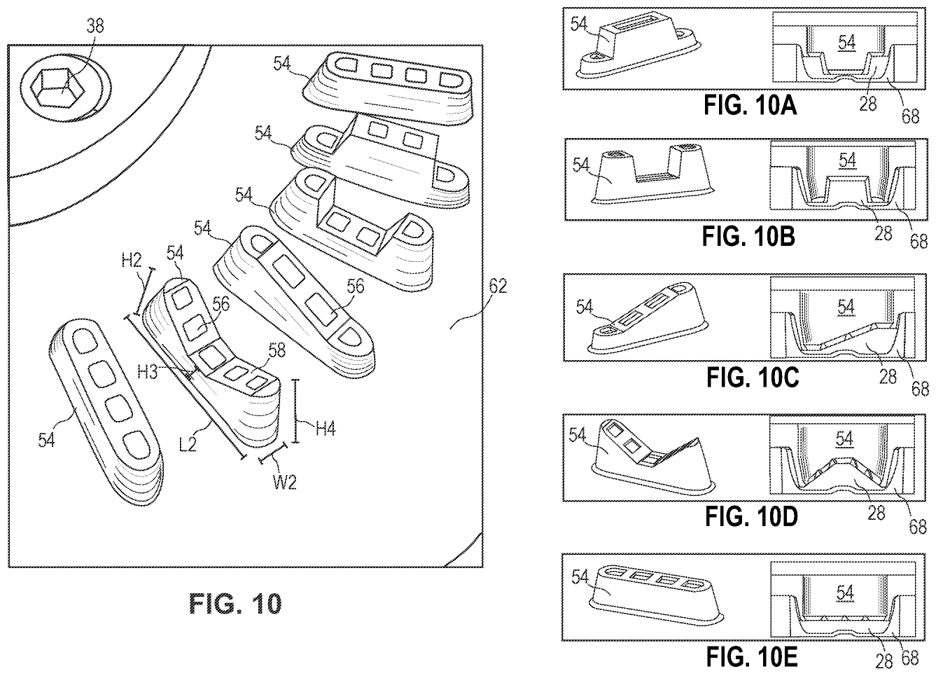

According to an example of the present disclosure, a filling head 20 for filling a container 22, such as a unit dose carrier, as shown in FIGS. 1-10E, is provided. As shown in FIG. 1, the filling head 20 includes a receptacle 24 having an interior surface 26 configured to receive a powder 28 and having a plurality of openings 34 disposed therein to allow passage of the powder 28. The receptacle 24 has an exterior surface 52 having a plurality of raised surfaces 54. Each of the plurality of raised surfaces 54 has a plurality of openings 56 aligned with at least one of the plurality of openings 34 of the interior surface 26 to allow passage of the powder 28 from the receptacle 24 to the container 22.

The dimensions of the filling head 20 allow it to be compatible with a filling machine's dimensions and with the container 22. The filling head 20 enables the container 22 to be filled with a defined amount of powder 28 and also allows the powder 28 to be layered in a linear or non-linear configuration. Further, the filling head 20 enables the powder 28 to be metered into a defined volume at a consistent density, to achieve weight control and uniformity of the powder 28.

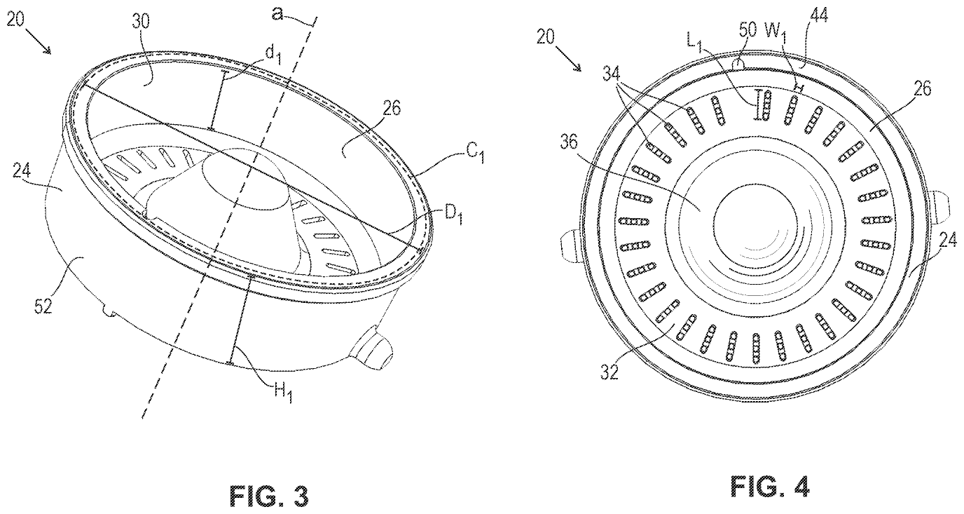

The filling head comprises the receptacle 24. The receptacle has a height HI and a diameter D1, as shown in FIG. 3. In some examples, the height HI is less than the diameter D1 of the receptacle. In some examples, the height HI is from about 1.5 centimeters (cm) to about 6 cm, from about 2 cm to about 4 cm, or from about 2 cm to about 3 cm. The height HI may be about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 cm. In some examples, the diameter D1 of the receptacle is from about 1.5 cm to about 8 cm, from about 2.5 cm to about 6 cm, or from about 3 cm to about 5 cm. The diameter D1 may be about 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0 cm.

The receptacle 24 comprises a circumference C1, as shown in FIG. 3. In some examples, the circumference C1 is from about 4 to about 26 cm, from about 6 to about 20 cm, or from about 10 to about 15 cm. In some examples, the circumference C1 may be about 4, 5, 6, 7, 8, 9, 10, 11, 12, 13, 14, 15, 16, 17, 18, 19, 20, 21, 22, 23, 24, 25 or 26 cm.

The receptacle 24 has an interior surface 26 configured to receive the powder 28. The interior surface 26 contains the powder 28 and provides a supply of the powder 28 to enable filling of the container 22. The interior surface 26 has a depth d1, as shown in FIG. 3. In some examples, the depth d1 is from about 1.0 centimeters (cm) to about 4 cm, from about 1.5 cm to about 3 cm, or from about 2 cm to about 3 cm. The depth d1 may be about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 or 4.0 cm.

The interior surface 26 defines a circumferential wall 30 and a floor 32. The floor 32 defines a plurality of openings 34. The plurality of openings 34 are configured for interaction with the powder 28 to allow passage of the powder 28 into the plurality of openings 34, out of a plurality of raised surfaces 54 that have a plurality of openings 56, the plurality of raised surfaces 54 defined by the exterior surface 52 of the receptacle 24, and into the container 22, as described herein. The plurality of openings 34 are circumferentially arrayed about a conical-shaped projection 36, as described herein.

In some examples, the plurality of openings 34 can include from about 1 to about 40 openings, from about 5 to about 35 openings, from about 10 to about 30 openings, from about 15 to about 25 openings, or from about 20 to about 22 openings. In some examples, the plurality of openings can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 openings.

The plurality of openings 34 may be any particular size that allows powder 28 to pass through during the filling process. The plurality of openings 34 may be slits and may be oval, rectangular, circular, and/or square shaped. In some examples, the plurality of openings may have a length LI, as shown in FIG. 4, of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, and/or 6.0 mm. In some examples, the plurality of openings may have a width W1, as shown in FIG. 4, of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and/or 2.0 mm.

As described above, the plurality of openings 34 are circumferentially disposed about the conical-shaped projection 36. The conical-shaped projection 36 is configured for fixed engagement substantially in a center of the floor 32 of the interior surface 26 via a fixation element, such as a screw 38. The floor 32 defines a first channel 40 disposed substantially in the center of the filling head 20, and an interior of the conical-shaped projection 36 defines a second interior channel 42. A longitudinal axis a extends through the first channel 40, as shown in FIG. 3. The screw 38 is configured to engage with the first channel 40 and the second interior channel 42 to lock the conical-shaped projection 36 in place. The conical-shape projection 36 may also be preformed into the floor 32 of the interior surface 26.

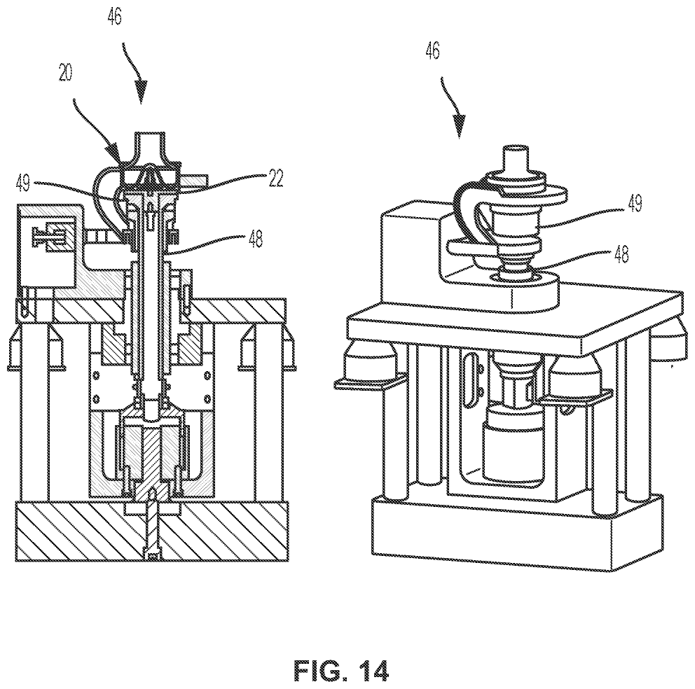

The circumferential wall 30 of the interior surface merges with an exterior surface 52 of the receptacle 24 and defines a ledge 44 that is disposed opposite the floor 32 of the interior surface 26. The ledge 44 is configured for engagement with a portion of a filling machine 46, as shown in FIG. 14. An indent 50 is defined from a portion of the ledge 44 and is configured for engagement with a portion of the filling machine 46.

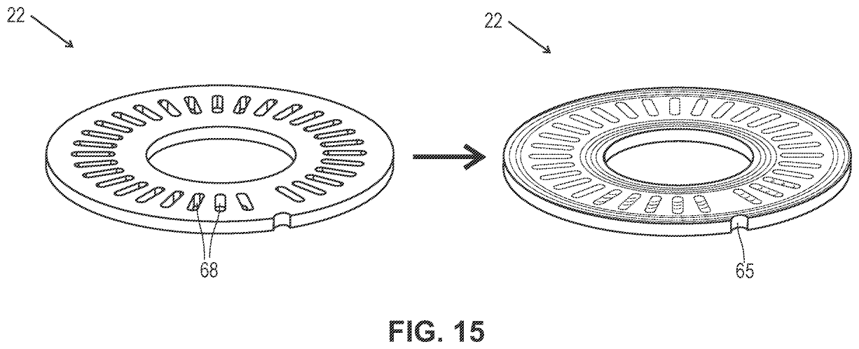

As described above, the receptacle 24 includes the exterior surface 52. The exterior surface 52 is configured for engagement with a container 22, such as a unit dose carrier, as shown in FIG. 15. For example, the unit dose carrier can be similar to the containers or inhaler discs disclosed in U.S. Pat. No. 9,399,103, which is hereby incorporated by reference herein in its entirety.

The exterior surface 52 of the receptacle 24 defines a plurality of raised surfaces 54. Each of the plurality of raised surfaces 54 has a plurality of openings 56 aligned with at least one of the plurality of openings 34 of the interior surface 26 to allow passage of the powder 28 from the receptacle 24 to the container 22. The plurality of raised surfaces 54, along with the plurality of openings 56, are circumferentially arrayed about the exterior surface 52 of the receptacle 24. In some examples, the plurality of raised surfaces 54 are circumferentially arrayed about a circular raised portion 57. The circular raised portion 57 is configured for engagement with a portion of the container 22.

In some examples, the plurality of raised surfaces 54 can include from about 1 to about 40 raised surfaces, from about 5 to about 35 raised surfaces, from about 10 to about 30 raised surfaces, from about 15 to about 25 raised surfaces, or from about 20 to about 22 raised surfaces. In some examples, the raised surfaces can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 raised surfaces.

The plurality of raised surfaces 54 have an inverted cone shape. However, the plurality of raised surfaces 54 may have variously configured shapes, as shown in FIGS. 10-10E. The plurality of raised surfaces 54 may have an egg tooth shape (e.g., FIG. 10A), a castle shape (e.g., FIG. 10B), an angled shape (e.g., FIG. 10C), an inverted cone shape (e.g., FIG. 10D), and/or a linear shape (e.g., FIG. 10E). The plurality of raised surfaces 54 may have a linear configuration or a non-linear or angled configuration.

The dimensions of the plurality of raised surfaces 54 are configured so that they can engage the container 22 without inhibiting or reducing powder flow. For example, the dimensions of the plurality of raised surfaces 54 can reduce the amount of powder 28 that would stick to the plurality of raised surfaces 54 when the plurality of raised surfaces 54 engage with pockets 68 of the container 22.

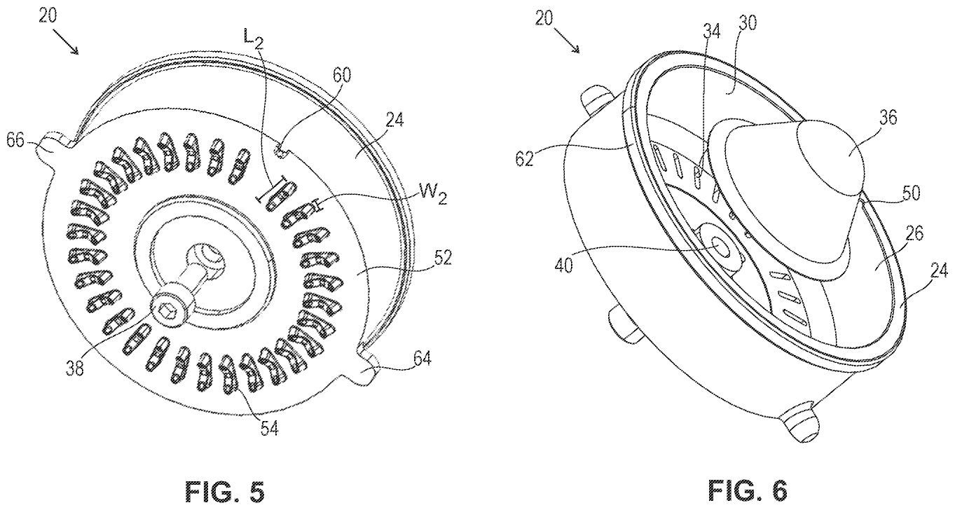

The plurality of raised surfaces 54 may have particular dimensions, as shown in FIGS. 5 and 10. In some examples, the plurality of raised surfaces 54 may have a length L2 of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, and/or 6.0 mm. In some examples, the plurality of raised surfaces 54 may have a width W2 of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and/or 2.0 mm. The plurality of raised surfaces 54 may have a height H2, H3 and H4. Heights H2, H3 and H4 may be from about 0.01 to about 5 mm, from about 0.5 to about 3 mm, or from about 1 to about 2 mm. In some examples, heights H2, H3 and/or H4 may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and/or 5.0 mm. In one example, heights H2 and H4 are equal, and height H3 is less than heights H2 and H4.

The plurality of raised surfaces 54 each have an upper surface 58 that defines the plurality of openings 56, and the upper surface 58 is configured for engagement with a container 22. Each of the plurality of raised surfaces 54 can have from about 1 to about 10 openings 56. In some examples, each of the plurality of raised surfaces 54 can have at least 1, 2, 3, 4, 5, 6, 7, 8, 9 and/or 10 openings 56 to allow passage of the powder 28 from the receptacle 24 to the container 22.

The plurality of openings 56 may be variously shaped, including but not limited to circular, square, rectangular, crescent, hexagonal, triangular, oval, slit, and/or star shaped. The plurality of openings 56 may have curved edges or straight edges. The plurality of openings 56 may be configured on the upper surface 58 of the plurality of raised surfaces 54 in a single row or multiple rows, as shown in FIG. 10.

The plurality of openings 56 may have a particular size. The dimensions of the plurality plurality of openings 56 can be any size that allows powder to pass through them during the filling process. The size of the plurality of openings 56 may be from about 0.01 to about 2 mm, from about 0.05 to about 0.5, or from about 0.08 to about 0.3 mm. In some examples, the size of the plurality of openings 56 may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9 and/or 2 mm.

The exterior surface 52 of the receptacle 24 defines a protuberance 60 that is disposed on an edge of the exterior surface 52 of the receptacle 24. The protuberance 60 is configured for engagement with a portion of the container 22, such as an indent 65 on the container 22 to facilitate engagement of the exterior surface 52 of the receptacle 24 with the container 22.

The exterior surface 52 of the receptacle 24 defines a threaded portion 62 adjacent to the ledge 44 that is configured for engagement with a portion of the filling machine 46. Alternatively, the threaded portion 62 can be smooth and not threaded. The exterior surface 52 defines a first flange 64 and a second flange 66, the first flange 64 being located opposite the second flange 66. The first flange 64 and the second flange 66 may be trunnions which allow the filling head 20 to pivot.

The container 22 that is filled by the filling head 20 comprises a plurality of pockets 68, as shown in FIG. 15, that are configured for disposal and storage of the powder 28. The container 22 further includes a covering disposed over at least the plurality of pockets 28, the covering configured for piercing engagement such that the powder 28 can be dispensed from the container 22.

Each of the plurality of pockets 68 is configured to a particular size to receive at least one of the plurality of raised surfaces 54, as shown in FIGS. 10A-10E. The plurality of pockets 68 are disposed radially on the container 22, each pocket 68 being sized to receive at least one of the plurality of raised surfaces 54 of the exterior surface 52 of the receptacle 24 to allow filling of each of the pockets 68 with the powder 28. The powder 28 that is dispensed by the filling head 20 is for a dry powder inhaler and can comprise a medicament and/or an excipient, as described herein. The filling head 20 can dispense about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, or 25 mg of powder 28, or any increments therebetween, out of each of the plurality of raised surfaces 54 and into each of the pockets 28. The amount of powder 28 that can be dispensed into each of the plurality of pockets 68 depends on the size of each of the pockets 68, as well as the amount of available pocket volume in each of the pockets 68.

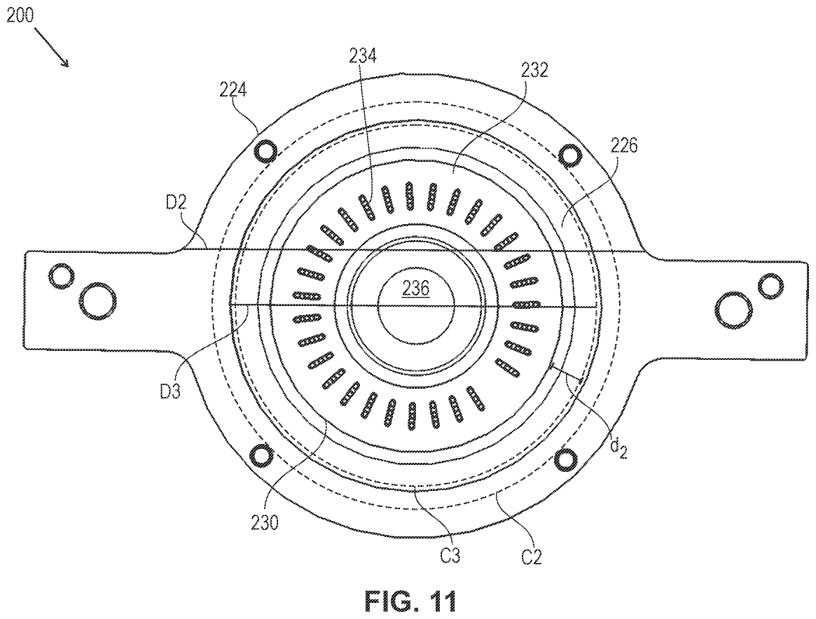

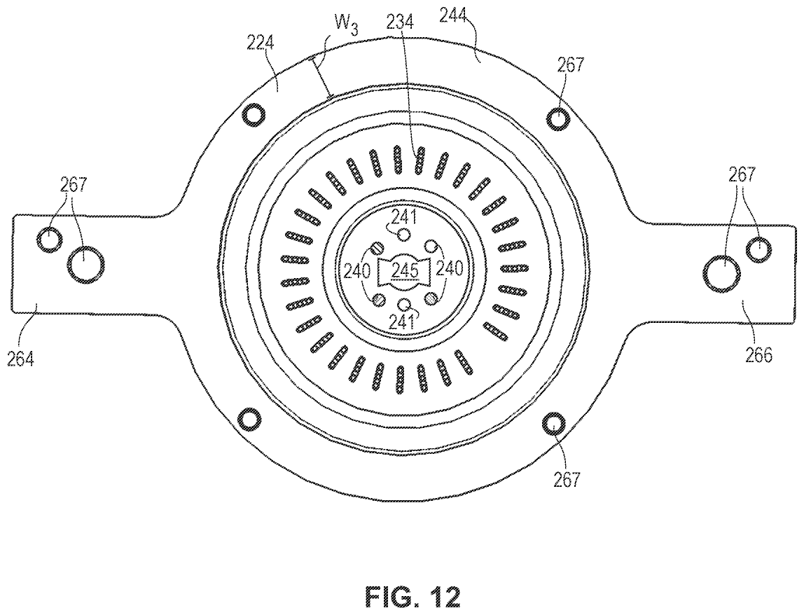

Referring to FIGS. 11-13, a filling head 200, similar to filling head 20 described above and shown in FIGS. 1-10E, is provided for filling container 22, such as a unit dose carrier. Like filling head 20, filling head 200 enables the container 22 to be filled with a defined amount of powder 28 and also allows the powder 28 to be layered in a linear or non-linear configuration. As shown in FIG. 11, the filling head 200 includes a receptacle 224 having an interior surface 226 configured to receive a powder 28 and having a plurality of openings 234 disposed therein to allow passage of the powder 28. The receptacle 224 has an exterior surface 252 having a plurality of raised surfaces 254. Each of the plurality of raised surfaces 254 have a plurality of openings 256 aligned with at least one of the plurality of openings 234 of the interior surface 226 to allow passage of the powder 28 from the receptacle 224 to the container 22.

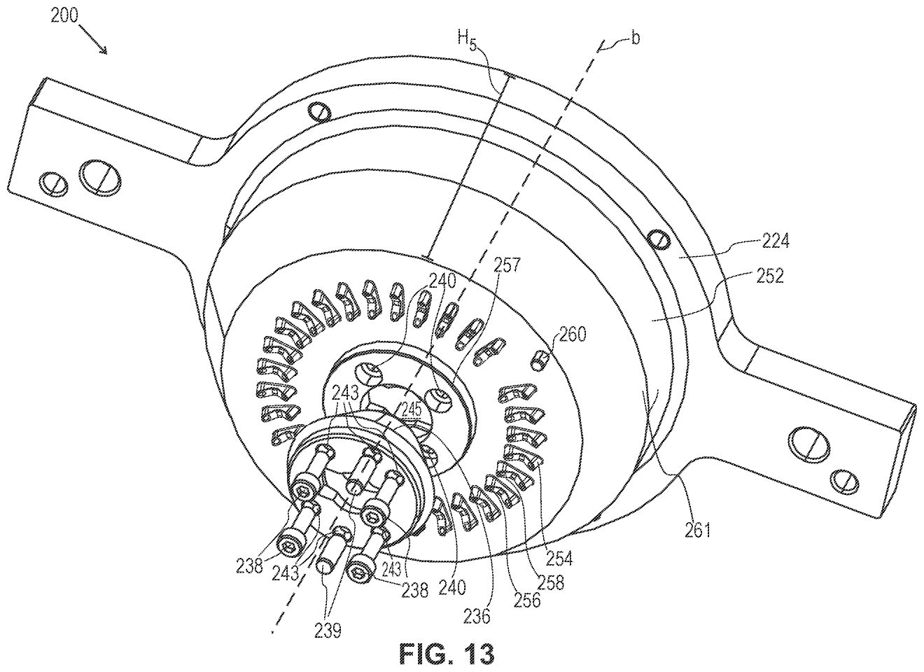

The filling head includes a receptacle 224. The receptacle has a height H5, as shown in FIG. 13, an outer diameter D2 and an inner diameter D3, as shown in FIG. 11. In some examples, the height H5 is less than diameters D2 and D3 of the receptacle. In some examples, the height H5 is from about 2 centimeters (cm) to about 8 cm, from about 4 cm to about 6 cm, or from about 3 cm to about 5 cm. The height H5 may be about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8 cm. In some examples, the outer or inner diameters D2, D3 of the receptacle are from about 1.5 cm to about 9 cm, from about 2.5 cm to about 6 cm, or from about 3 cm to about 5 cm. The diameters D2 and D3 may be about 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9 cm.

The receptacle 224 comprises an outer circumference C2 and an inner circumference C3, as shown in FIG. 11. In some examples, circumferences C2, C3 are from about 4 to about 29 cm, from about 6 to about 20 cm, or from about 10 to about 15 cm. In some examples, the circumferences C2 and C3 may be about 4, 5, 6, 7, 8, 9, 10, 11, 12, 13, 14, 15, 16, 17, 18, 19, 20, 21, 22, 23, 24, 25, 26, 27, 28 or 29 cm.

The receptacle 224 has an interior surface 226 configured to receive powder 28. The interior surface 226 contains the powder 28 and provides a continuous supply of the powder 28 to enable filling of the container 22. The interior surface has a depth d2, as shown in FIG. 11. In some examples, the depth d2 is from about 1.0 centimeters (cm) to about 6 cm, from about 1.5 cm to about 3 cm, or from about 2 cm to about 3 cm. The depth d2 may be about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 or 6 cm.

The interior surface 226 defines a circumferential wall 230 and a floor 232. The floor 232 defines a plurality of openings 234, which can be identical to the plurality of openings 34, as described above. The plurality of openings 234 are configured for interaction with the powder 28 to allow passage of the powder 28 into the plurality of openings 234, out of a plurality of raised surfaces 254 that have a plurality of openings 256, the plurality of raised surfaces 254 defined by the exterior surface 252, and into the container 22, as described herein. The plurality of openings 234 are circumferentially arrayed about a conical-shaped projection 236, as described herein.

In some examples, the plurality of openings 234 can include from about 1 to about 40 openings, from about 5 to about 35 openings, from about 10 to about 30 openings, from about 15 to about 25 openings, or from about 20 to about 22 openings. In some examples, the plurality of openings 234 can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 openings.

The plurality of openings 234 may be slits and may be oval, rectangular, circular, and/or square shaped. The plurality of openings 234 may be a particular size. In some examples, the plurality of openings 234 may have the same length LI and width W1, as described above regarding the plurality of openings 34.

As described above, the plurality of openings 234 are circumferentially disposed about the conical-shaped projection 236. The conical-shaped projection 236 is configured for fixed engagement substantially in a center of the floor 232 of the interior surface 226 via fixation element(s), such as screws 238 and dowel pins 239. The floor 232 defines channels 240, circular indents 241 and an opening 245 disposed substantially in the center of the filling head 200. An interior of the conical-shaped projection 236 defines an interior channel 242 and channels 243. A longitudinal axis b extends through opening 245, as shown in FIG. 13. Each of the screws 238 are configured to engage with one of the channels 240 of the floor 232 and one of the channels 243 of the conical-shaped projection 236, and each of the dowel pins 239 are configured to engage with one of the circular indents 241 of the floor 232 and one of the channels 243 of the conical-shaped projection 236 to lock or fix the conical-shaped projection 236 in place. The conical-shaped projection 236 may also be preformed into the floor 232 of the interior surface 226.

The circumferential wall 230 of the interior surface 226 merges with an exterior surface 252 of the receptacle 224 and defines a ledge 244 that is disposed opposite the floor 232 of the interior surface 226. The ledge 244 is configured for engagement with a portion of the filling machine 46, as shown in FIG. 14. The ledge 244 has a width W3. Width W3 may be from about 1 mm to about 6 mm, from about 1 mm to about 4 mm, or from about 1 mm to about 2 mm. The width W3 may be about 1, 2, 3, 4, 5, or 6 mm, or any increment therebetween.

As described above, the receptacle 224 includes the exterior surface 252. The exterior surface 252 is configured for engagement with a container 22, such as a unit dose carrier, as shown in FIG. 15. For example, the unit dose carrier can be similar to the containers or inhaler discs disclosed in U.S. Pat. No. 9,399,103.

The exterior surface 252 of the receptacle defines a plurality of raised surfaces 254. Each of the plurality of raised surfaces 254 has a plurality of openings 256 aligned with at least one of the plurality of openings 234 of the interior surface 226 to allow passage of the powder 28 from the receptacle 224 to the container 22. The plurality of raised surfaces 254 and the plurality of openings 256 may be identical to the plurality of raised surfaces 54 and the plurality of openings 56, as described above and relating to FIGS. 1-10E. The plurality of raised surfaces 254, along with the plurality of openings 256, are circumferentially arrayed about the exterior surface 252 of the receptacle 224. In some examples, the plurality of raised surfaces 254 are circumferentially arrayed about a circular raised portion 257. The circular raised portion 257 is configured for engagement with a portion of the container 22.

In some examples, the plurality of raised surfaces 254 can include from about 1 to about 40 raised surfaces, from about 5 to about 35 raised surfaces, from about 10 to about 30 raised surfaces, from about 15 to about 25 raised surfaces, or from about 20 to about 22 raised surfaces. In some examples, the plurality of raised surfaces 254 can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 raised surfaces.

The plurality of raised surfaces 254 have an inverted cone shape, as described above with regard to the plurality of raised surfaces 54 and relating to FIGS. 1-10E. However, the plurality of raised surfaces 254 may have variously configured shapes, as shown in FIGS. 10-10E. The plurality of raised surfaces may have an egg tooth shape (e.g., FIG. 10A), a castle shape (e.g., FIG. 10B), an angled shape (e.g., FIG. 10C), an inverted cone shape (e.g., FIG. 10D), and/or a linear shape (e.g., FIG. 10E). The plurality of raised surfaces may have a linear configuration or a non-linear or angled configuration.

The plurality of raised surfaces 254 may have particular dimensions, as shown in FIG. 10, that are the same as the dimensions of the plurality of raised surfaces 54, as described above.

The plurality of raised surfaces 254 each have an upper surface 258 that defines the plurality of openings 256, and the upper surface 258 is configured for engagement with a container 22. Each of the plurality of raised surfaces 254 can have from about 1 to about 10 openings 256. In some examples, each of the plurality of raised surfaces 254 can have at least 1, 2, 3, 4, 5, 6, 7, 8, 9 and/or 10 openings 256 to allow passage of the powder 28 from the receptacle 224 to the container 22.

The plurality of openings 256 may be variously shaped, including but not limited to circular, square, rectangular, crescent, hexagonal, triangular, oval, slit, and/or star shaped. The plurality of openings 256 may have curved edges or straight edges. The plurality of openings 256 may be configured on the upper surface 258 of the plurality of raised surfaces 254 in a single row or multiple rows, as shown in FIG. 10. The plurality of openings 256 may have a particular size, similar or identical to the size of the plurality of openings 56, as shown in FIG. 10.

The exterior surface 252 of the receptacle 224 defines a protuberance 260 that is disposed on an edge of the exterior surface 252 of the receptacle 224. The protuberance 260 is configured for engagement with a portion of the container 22, such as indent 65 to facilitate engagement of the exterior surface 252 of the receptacle 224 with the container 22.

The exterior surface 252 of the receptacle 224 defines a beveled portion 261 that defines a part of the shape of the exterior surface 252 of the receptacle 224. Alternatively, the beveled portion 261 can be smooth and not beveled. The exterior surface 252 defines a first flange 264 and a second flange 266, the first flange 264 being located opposite the second flange 266. The first flange 264 and the second flange 266 can be formed monolithically with the ledge 244 of the exterior surface 252. The ledge 244, the first flange 264 and the second flange 266 can have a plurality of holes 267 of the same or different diameter that are configured for engagement with a portion of the filling machine 46.

The container 22 that is filled by the filling head 200 comprises a plurality of pockets 68, as shown in FIG. 15, that are configured for disposal and storage of the powder 28. The container 22 further includes a covering disposed over at least the plurality of pockets 68, the covering configured for piercing engagement such that the powder 28 can be dispensed from the container 22.

The filling head 200 can dispense about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, or 25 mg of powder, or any increments therebetween, out of each of the plurality of raised surfaces 254 and into the pockets 68. The amount of powder 28 that can be dispensed into each of the plurality of pockets 68 depends on the size of each of the pockets 68, as well as the amount of available pocket volume in each of the pockets 68.

### Filling Head Materials

The components of the filling head 20, 200 can be fabricated from materials suitable for medical applications, including metals, synthetic polymers, and/or ceramics. For example, the components of the filling head 20, 200, individually or collectively, can be fabricated from materials such as stainless steel alloys, aluminum, commercially pure titanium, titanium alloys, Grade 5 titanium, super-elastic titanium alloys, cobalt-chrome alloys, super-elastic metallic alloys (e.g., Nitinol, super elasto-plastic metals, such as GUM METAL®), ceramics and composites thereof, thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, composites of PEEK, or other resorbable polymers such as polyaetide, polyglycolide, polytyrosine carbonate, polycaroplaetohe, and their combinations.

Various components of the filling head 20, 200 may have material composites, including the above materials, to achieve various desired characteristics such as strength, rigidity, elasticity, compliance, biomechanical performance, and durability. The components of the filling head 20, 200, individually or collectively, may also be fabricated from a heterogeneous material such as a combination of two or more of the above-described materials. The components of the filling head 20, 200 may be monolithically formed, integrally connected, and/or include fixation elements, as described herein.

### Medicaments

The filling head 20, 200 can fill the container 22 with a variety of powder APIs, powder excipients, and/or any other powder in micronized form. The powder should have the particle size and flowability to be administered by inhalation (e.g., oral or nasal inhalation). The API can be any API suitable for inhalation including an anticholinergic agent, a bronchodilator, and/or corticosteroids.

The anticholinergic agents include but are not limited to trihexyphenidyl, benztropine mesylate, ipratropium, tiotropium, orphenadrine, atropine, flavoxate, oxybutynin, scopolamine, hyoscyamine, tolterodine, belladonna alkaloids, fesoterodine, solifenacin, darifenacin, propantheline, 5-[3-(3-Hydroxyphenoxy)azetidin-1-yl]-5-methyl-2,2-diphenylhexanamide or a combination thereof. The bronchodilators include but are not limited to adrenergic bronchodilators, including but not limited to levalbuterol, metaproterenol, pirbuterol, formoterol, terbutaline, or albuterol; anticholinergic bronchodilators, including but not limited to aclidinium systemic, ipratropium systemic, tiotropium systemic, orumeclidinium systemic; bronchodilator combinations, including but not limited to umeclidinium/vilanterol systemic, budesonide/formoterol systemic, fluticasone/salmeterol systemic, albuterol/ipratropium systemic, fluticasone/vilanterol systemic, olodaterol/tiotropium systemic, formoterol/mometasone systemic, formoterol/glycopyrrolate systemic, orglycopyrrolate/indacaterol systemic; and methylxanthines, including but not limited to theophylline systemic, aminophylline systemic, or dyphylline systemic. The corticosteroids include but are not limited to beclomethasone, budesonide, flunisolide, fluticasone, mometasone, ciclesonide or tiotropium.

The powder may also include substances traditionally regarded as actives, drugs and bioactive agents, as well as biopharmaceuticals (e.g., peptides, hormones, nucleic acids, gene constructs, etc.) typically employed to treat a number of conditions which are defined broadly to encompass diseases, disorders, infections, and the like. Exemplary APIs, without limitation, include antibiotics, antivirals, H2-receptor antagonists, 5HT1 agonists, 5HT3 antagonists, COX2-inhibitors, APIs used in treating psychiatric conditions such as depression, anxiety, bipolar condition, tranquilizers, APIs used in treating metabolic conditions, anticancer APIs, APIs used in treating neurological conditions such as epilepsy and Parkinson's Disease, respiratory conditions such as, for example, asthma, Chronic Obstructive Pulmonary Disease (COPD), bronchitis, bronchiolitis, emphysema, or the like. Also included are APIs used in treating cardiovascular conditions, non-steroidal anti-inflammatory APIs, APIs used in treating Central Nervous System conditions, and APIs employed in treating hepatitis.

In some examples, the APIs may also be selected from: analgesics, e.g., codeine, dihydromorphine, ergotamine, fentanyl or morphine; anginal preparations, e.g., diltiazem; antiallergics, e.g., cromoglycate (e.g. as the sodium salt), ketoprofen or nedocromil (e.g., as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g., as the dipropionate ester), fluticasone (e.g., as the propionate ester), flunisolide, prednisone, prednisolone, budesonide, rofleponide, mometasone (e.g., as the furoate ester), ciclesonide, triamcinolone (e.g., as the acetonide), or 6a,9a-difluoro-1:1β-hydroxy-16a-methyl-3-oxo-17a-propionyloxy-androsta-:1,4-diene-17P-carbothioic acid S-(2-oxo-tetrahydro-furan-3-yl)ester; antitussives, e.g., noscapine; bronchodilators, e.g., albuterol (e.g., as free base or sulphate), salmeterol (e.g., as xinafoate), ephedrine, adrenaline, fenoterol (e.g., as hydrobromide), formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g., 2R,3R,4S,5R)-2-[6-Amino-2-(1S-hydroxymethyl-2-phenyl-ethylamino)purin-9-yl]-5-(2-ethyl-2H-tetrazol-5-yl)-tetrahydro-furan-3,4-diol (e.g. as maleate); a4 integrin inhibitors, e.g., (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino]propanoic acid (e.g., as free acid or potassium salt), diuretics, e.g., amiloride; anticholinergics, e.g., ipratropium (e.g., as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; or vaccines, diagnostics, and gene therapies. It will be clear to a person skilled in the art that, where appropriate, the APIs may be used in the form of salts, (e.g., as alkali metal or amine salts or as acid addition salts) or as esters (e.g., lower alkyl esters) or as solvates (e.g., hydrates) to optimize the activity and/or stability of the API.

Additionally, the APIs may be selected from, for example, antibiotics. Such antibiotics include, for example, nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. In the following examples of such antibiotics are listed: ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, or methyl-2-pentylphenol. The active antibiotics could be in standard forms or used as salts, hydrates, or esters etc. A combination of two or more of the above-listed drugs may be used. The antibiotics can be clarithromycin, erythromycin, roxithromycin, azithromycin, amoxicillin, metronidazole, tinidazole, or tetracycline. Clarithromycin and metronidazole alone or in combination are especially suitable.

Moreover, APIs may also be selected from, for example, antivirals. Examples of APIs that are effective for the treatment of viral and viral associated conditions are (1-alpha, 2-beta, 3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine(oxetanocin-G), acyclic nucleosides, for example acyclovir, valaciclovir, famciclovir, ganciclovir, and penciclovir, acyclic nucleoside phosphonates, for example (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid(tenofovir), and (R)-[[2-(6-Amino-9H-purin-9-yl)-1-methylethoxy]methyl]phosphonic acid bis-(isopropoxy carbonyloxymethyl) ester (bis-POC-PMPA), ribonucleotide reductase inhibitors, for example 2-acetylpyridine 5-[(2-chloroanilino)thiocarbonyl)thiocarbonohydrazone and hydroxyurea, nucleoside reverse transcriptase inhibitors, for example 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddl, didanosine), 2′,3′-didehydrothymidine (d4T, stavudine), (−)-beta-D-2,6-diaminopurine dioxolane (DAPD), 3′-azido-2′,3′-dideoxythymidine-5′-H-phosphophonate (phosphonovir), 2′-deoxy-5-iodo-uridine(idoxuridine), (−)-cis-1-(2-hydroxymethyl)-1,3-oxathiolane 5-yl)-cytosine (lamivudine), cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol (abacavir), 9-[4-hydroxy-2-(hydroxymethyl) but-1-yl]-guanine (H2 G), ABT-606 (2HM-H2G) and ribavirin, protease inhibitors, for example indinavir, ritonavir, nelfmavir, amprenavir, saquinavir, (R)—N-tert-butyl-3-[(2S,3S)-2-hydroxy-3-N—[(R)-2-N-(isoquinolin-5-yloxyacetyl)amino-3-methylthio-propanoyl]amino-4-phenylbutanoyl]-5,5-dimethyl-1,3-thiazolidine-4-carboxamide (KNI-272), 4R-(4alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate(mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)—[N-(methoxycarbonyl)-I-tert-leucylamino]-4-phenylbutyl-Nalpha-methoxycarbonyl)-N′-[4-(2-pyridyl)benzyl]-L-tert-leucylhydrazide (BMS-232632), 3-(2 (S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-hydroxy-1(S)-indanyl)-2(R)-phenyl-methyl-4 (S)-hydroxy-5-(1-(1-(4-benzo[b]furanylmethyl)-2(S)—N′-(tert-butylcarboxamido)piperazinyl)pentanamide (MK-944A), and (3S)-tetrahydrofuran-3-yl (1S,2R)-[[(4-aminophenyl)sulphonyl)](isobutyl)amino]-1-benzyl-2-(phosphonooxy)propylcarbamate monocalcium salt(fosamprenavir), interferons such as a-interferon, renal excretion inhibitors such as probenecid, nucleoside transport inhibitors such as dipyridamole; pentoxifylline, N-acetylcysteine (NAC), Procysteine, a-trichosanthin, phosphonoformic acid, as well as immunomodulators such as interleukin II or thymosin, granulocyte macrophage colony stimulating factors, erythropoetin, soluble CD4 and genetically engineered derivatives thereof, non-nucleoside reverse transcriptase inhibitors (NNRTIs), for example nevirapine (BI-RG-587), alpha-((2-acetyl-5-methylphenyl)amino)-2,6-dichloro-benzeneacetamide (loviride), 1-[3-(isopropylamino)-2-pyridyl]4-[5-(methanesulfonamido)-1H-indol-2-ylcarbonyl]piperazine monomethanesulfonate (delavirdine), (10R, 11S, 12S)-12-Hydroxy-6,6,10,11-tetramethyl-4-propyl-11,12-dihydro-2H, 6H, 10H-benzo (1, 2-b:3, 4-b′:5, 6-b″)tripyran-2-one ((+) calanobde A), (4S)-6-Chloro-4-[1E)-cyclopropylethenyl)-3,4-dihydro-4-(trifluoromethyl)-2(1H)-quinazobnone (DPC-083), (S)-6-chloro-4-(cyclopropylethynyl)-1,4-dihydro4-(trifluoromethyl)-2H-3,1-benzoxazin-2-one(efavirenz, DMP 266), 1-(ethoxymethyl)-5-(1-methylethyl)-6-(phenylmethyl)-2,4(1H,3H)-pyrimidinedione (MKC-442), and 5-(3,5-dichlorophenyl)thio-4-isopropyl-1-(4-pyridyl)methyl-1H-imidazol-2-ylmethyl carbamate(capravirine), glycoprotein 120 antagonists, for example PRO-2000, PRO-542 and 1,4-bis[3-[(2,4-dichlorophenyl)carbonylamino]-2-oxo-5,8-disodiumsulfanyl]naphthalyl-2,5-dimethoxyphenyl-1,4-dihydrazone (FP-21399), cytokine antagonists, for example reticulose (Product-R), 1,1′-azobis-formamide (ADA), 1,11-(1,4-phenylenebis(methylene))bis-1,4,8,1 1-tetraazacyclotetradecane octahydrochloride (AMD-3100), integrase inhibitors, for example, S-1360, and fusion inhibitors.

The powder may also include pharmaceutically acceptable salts, esters, solvates, and/or hydrates of the pharmaceutically active substances referred to hereinabove. Various combinations of any of the above powders may also be employed.

The powder may also include excipients such as glucose, arabinose, lactose, sucrose, maltose, mannitol, dextrans, magnesium stearate, or a combination thereof. The excipient may include but is not limited to monosaccharides such as galactose, mannose, sorbose; disaccharides such as lactose, sucrose and trehalose and the like; polysaccharides such as starch, raffinose, dextran and the like; and mixtures thereof.

### Methods

The present disclosure also provides a method of filling a unit dose carrier with a filling head 20, 200, the method comprising: attaching the filling head 20, 200 to a filling machine 46, the filling head 20, 200 comprising a receptacle 24, 224, the receptacle 24, 224 having an interior surface 26, 226 configured to receive a powder 28 and having a plurality of circumferentially arrayed openings 34, 234 disposed therein to allow passage of the powder 28; the receptacle 24, 224 having an exterior surface 52, 252, the exterior surface 52, 252 of the receptacle 24, 224 having a plurality of circumferentially arrayed raised surfaces 54, 254, each of the plurality of raised surfaces 54, 254 having a plurality of openings 56, 256 aligned with at least one of the plurality of openings 34, 234 of the interior surface 26, 226; loading the interior surface 26, 226 with the powder 28; engaging the plurality of circumferentially arrayed raised surfaces 54, 254 with a plurality of pockets 68 disposed in the unit dose carrier; attaching the unit dose carrier to the filling head 20, 200; administering a plurality of taps to the filling head 20, 200; and filling each of the plurality of the pockets 68 with at least a first layer of powder 28.

In some examples, after the interior surface 26, 226 is loaded, the powder 28 flows through the plurality of circumferentially arrayed openings 34, 234, out of the plurality of openings 56, 256 of the plurality of circumferentially arrayed raised surfaces 54, 254 and into each of the plurality of pockets 68 of the unit dose carrier.

In some examples, (i) the plurality of circumferentially arrayed raised surfaces 54, 254 engage the plurality of pockets 68 to dispose a defined volume amount of powder 28 into the plurality of pockets 68; (ii) the filling head 20, 200 disposes at least a first layer, a second layer, a third layer, a fourth layer, and/or a fifth layer of the powder 28 into each of the plurality of pockets 68; or (iii) the filling head 20, 200 fills each of the plurality of pockets 68 with the powder 28 in a total amount from about 2 μ [symbol] to about 32 μ [symbol] .

In some examples, the filling head 20, 200 can be compatible with a filling machine 46, as shown in FIG. 14.

In some examples, a method of filling the filling head 20 and/or the filling head 200 is provided. The filling head 20, 200 is loaded with a controlled quantity of powder 28 to provide a reservoir of material to be filled. According to the well-recognized principle of bridging or arching, as described in Shrimpton et al, Powder Technology, 249, pp. 24-37 (2013), which is hereby incorporated by reference herein in its entirety, the powder 28 in the filling head 20, 200 forms bridges across the plurality of openings 34, 234 in the interior surface 26, 226 of the receptacle 24, 224 of the filling head 20, 200, which prevents the powder 28 from flowing through the plurality of openings 34, 234 under static conditions. During the filling operation, after the empty container 22 (e.g., container as a disc, as shown in FIG. 15) is clamped to the underside of the filling head 20, 200, such as through the use of clamping cylinder 49, the filling machine 46 undergoes a defined number of vertical taps. The controlled impact from each tap breaks the powder bridges, allowing a quantity of powder 28 to move through the plurality of openings 34, 234, out of the plurality of raised surfaces 54, 254 having a plurality of openings 56, 256, and into each of the plurality of pockets 68 of the container 22 (e.g., disc). The impact also consolidates the powder 28 within each of the plurality of pockets 68. The number of taps is controlled to give the correct fill weight desired. The filled container 22 (e.g., disc) is unclamped from the filling head 20, 200 when the defined number of taps are completed. The number of taps from the machine 46 and filling head 20, 200 subflush (e.g., the engagement of the plurality of raised surfaces 54, 254 with the container 22 and powder 28) are parameters that control the fill weight of the powder 28. The number of taps are defined as the number of impacts in the filling cycle for each container 22 (e.g., disc). Increasing the number of taps leads to an increased fill weight by increasing the density of the powder 28 in the each of the filled pockets 68. The number of taps per container 22 (e.g., disc) is a variable defined by the operator during manufacture to achieve the target fill weight.

Additional filling machine parameters can be used to control the fill weight of the powder, including impact velocity and downward acceleration. These additional parameters cause the tapping frequency and tapping height to vary. For example, an increase in the impact velocity at a fixed acceleration will require the tapping column 48 (FIG. 14) to move at a greater height and thus will take longer to achieve a higher velocity, and therefore, fewer taps per unit time will result.

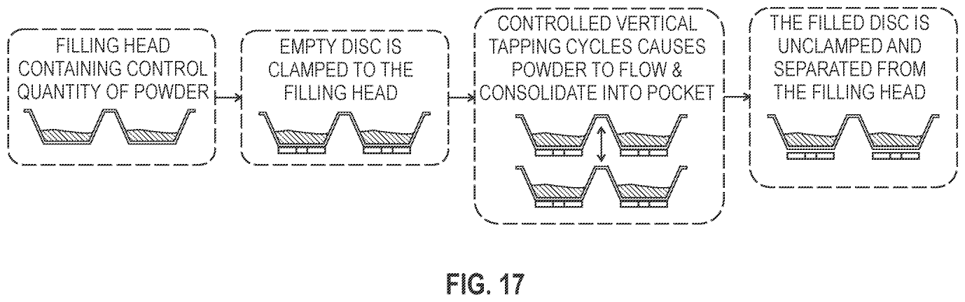

In some examples, filling heads 20 and 200 can use the same method of operating to dispense metered quantities of the powder 28 into individual doses in each of the plurality of pockets, as shown in FIG. 17.

In this method, the filling head 20, 200 is loaded with a controlled level of powder via the filling machine 46. The filling machine 46 clamps the container 22 (e.g., disc) to the filling head 20, 200, such as through the use of clamping cylinder 49. The filling head 20, 200 is then “tapped” to transfer powder 28 from the filling head 20, 200 into the each of the pockets of the container 22 (e.g., disc) to the target fill weight. The filled container 22 (e.g., disc) is then removed for sealing.

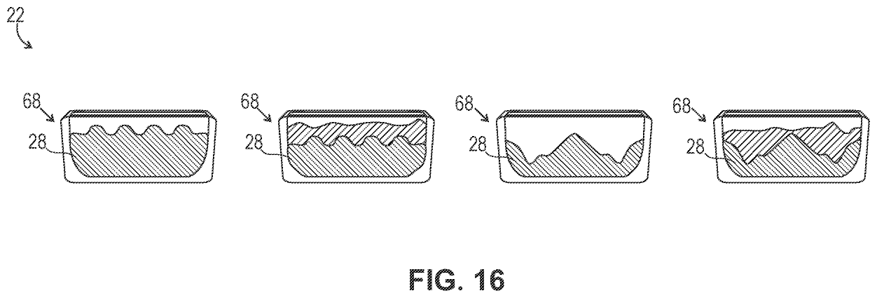

In some examples, filling head 20 and/or 200 can be used along or in conjunction in a method to layer powders 28 into each of the plurality of pockets 68 of the container 22. In some examples, the layers can be configured in various patterns such as, for example, in regular or irregular patterns, as shown in FIG. 16. In some examples, depending on the shape of each of the plurality of raised surfaces 54, 254, a portion or an entire layer or layers can be wavy, angled, arcuate, concave, convex, inclined, and/or declined. In some examples, depending on the shape of the each of the plurality of raised surfaces 54, 254, a portion or an entire layer or layers can be squared, circular, triangular, dimpled, toothed, inverted, flat, pentagonal, and/or hexagonal.

In some examples, the filling head 20 and/or 200 can be used alone or in conjunction in a method to create one or a plurality of layers within the plurality of pockets 68 of the container 28. The filling head 20 and/or 200 can create at least 1 to about 10 layers, at least 1 to about 8 layers, at least 1 to about 6 layers, at least 1 to about 4 layers, at least 1 to about 3 layers, or at least 1 to about 2 layers. The filling head 20 and/or 200 can create at least 1 to about 2, 3, 4, 5, 6, 7, 8, 9 or 10 layers.

### EXAMPLES

The examples below illustrate several contemplated filling head configurations, as well as the low fill weight layering capability of the filling head.

### Example 1: Filling Head Configurations

The filling head can have various configurations, including but not limited to the configurations shown in Table 1 below.

TABLE 1

| | Available Pkt. | Nominal Fill Wt. |
|---|---|---|
| Filling head Description | Vol (μl) | (mg) |
| 9.1 μl inverted cone, 3 hole, | 9.10 | 7.0 |
| 1.1 mm slot width | | |
| 9.1 μl inverted cone, 5 hole | 9.10 | 7.0 |
| 7 mg fill wt - MRG002; | 9.10 | 7.0 |
| (alternate pad) | | |
| 7 mg fill wt - MRG002; | 9.10 | 7.0 |
| (grouped pad) | | |
| 0.35 mm sub-flush, 4 hole | 26.43 | 19.8 |
| 0.45 mm sub-flush, 4 hole | 25.22 | 18.9 |
| 0.55 mm sub-flush, 4 hole | 24.01 | 18.0 |
| 0.96 mm sub-flush, 4 hole | 19.04 | 14.3 |
| 1.08 mm sub-flush, 4 hole | 17.59 | 13.2 |
| 1.20 mm sub-flush 4 hole | 16.14 | 12.1 |
| 9.1 μl inverted cone, 3 hole, | 9.10 | 7.0 |
| 1.1 mm slot width | | |
| 10.0 μl inverted cone, 3 hole, | 10.00 | 7.7 |
| 1.1 mm slot width | | |
| 8.2 μl inverted cone, 5 hole, | 8.20 | 6.3 |
| low trunnion | | |
| 9.1 μl inverted cone, 5 hole, | 9.10 | 7.0 |
| low trunnion | | |
| 10.0 μl inverted cone, 5 hole, | 10.00 | 7.7 |
| low trunnion | | |
| 11.0 μl inverted cone, 5 hole, | 11.00 | 8.5 |
| low trunnion | | |

* The term “hole” is used to describe the plurality of holes defined in the plurality of raised surfaces on the exterior surface of the filling head. Further, the “nominal fill weight” is dependent on the physical properties of the powder being filled.

### Example 2: Low Fill Weight Layering Capability of the Filling Head

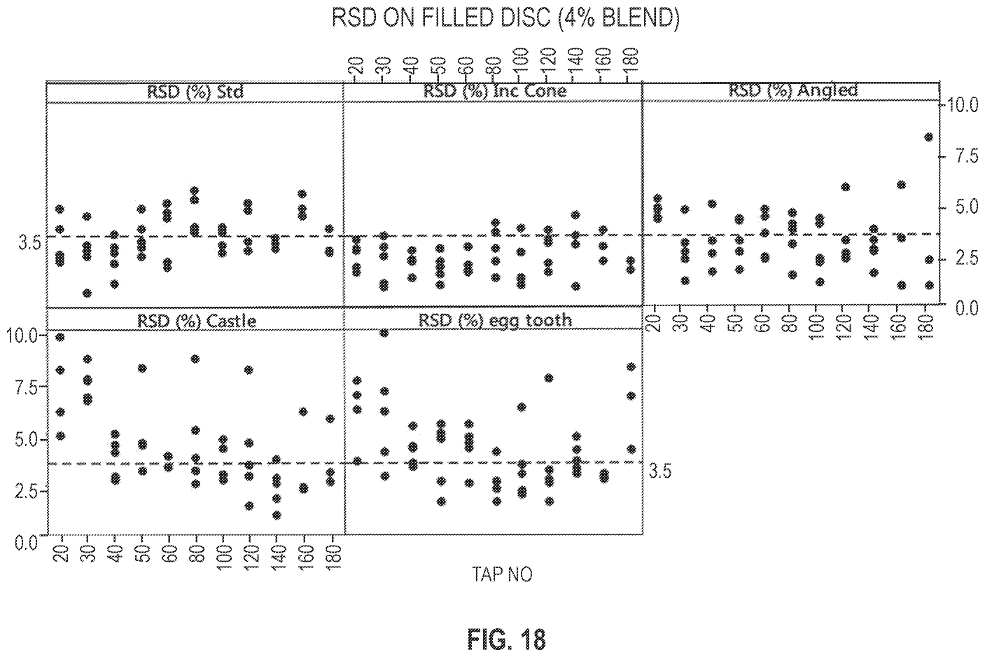

This example compared the plurality of raised surfaces shapes shown in FIGS. 10A-10E and determined what shape would provide a lower fill weight variability (RSD) when filling a pocket of a container with layers of powder. FIG. 18 provides a chart of the results of a comparison between different pluralities of raised surfaces shapes, and how these shapes performed when a lower fill weight of powder was desired. As depicted in FIG. 18, results showed that an inverted cone shape, as shown in FIG. 10D performed the best when a lower fill weight was desired. The inverted cone shape provides a relatively small cross-sectional area (or volume) of the powder last filled into the pocket when compared to the powder height. It was also determined that it is beneficial to fill the last powder (layer) into the pocket within a relatively small volume so that the small variations in the powder height do not cause a large change in the powder volume, which would lead to a large change (variation) in fill weight.

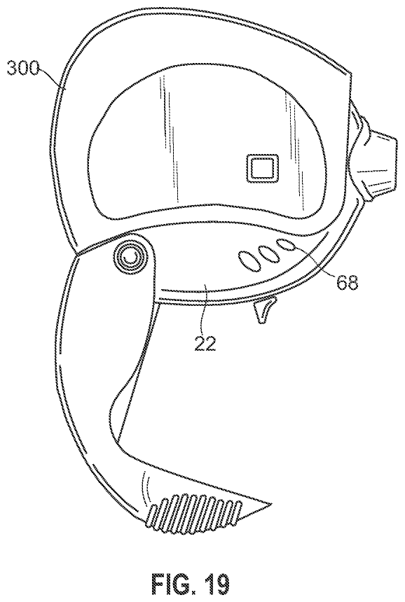

FIG. 19 illustrates an example of an assembled dry powder inhaler 300 having a container 22 shown as a disc having pockets 68 filled with a powder containing a medicament. The filling head can be used to fill the pockets with the powder containing the medicament, where the disc can be loaded into the dry powder inhaler and a dose of medicament can be inhaled by the patient via the dry powder inhaler.

It is to be understood that the invention may assume various alternative variations and step sequences, except where expressly specified to the contrary. It is also to be understood that the specific devices and processes illustrated in the attached drawings and described in the specification are simply exemplary embodiments or aspects of the invention. Although the invention has been described in detail for the purpose of illustration based on what is currently considered to be the most practical and preferred embodiments or aspects, it is to be understood that such detail is solely for that purpose and that the invention is not limited to the disclosed embodiments or aspects, but, on the contrary, is intended to cover modifications and equivalent arrangements that are within the spirit and scope thereof. For example, it is to be understood that the present invention contemplates that to the extent possible, one or more features of any embodiment or aspect can be combined with one or more features of any other embodiment or aspect.

1. A filling head for filling a container, the filling head comprising: a receptacle, the receptacle comprising: an interior surface configured to receive a powder and having a plurality of openings disposed therein to allow passage of the powder; and an exterior surface, the exterior surface of the receptacle having a plurality of raised surfaces, each of the plurality of raised surfaces having a plurality of openings aligned with at least one of the plurality of openings of the interior surface to allow passage of the powder from the receptacle to the container.

2. The filling head according to claim 1, wherein the plurality of openings are circumferentially arrayed about the interior surface and the plurality of openings of the raised surfaces are circumferentially arrayed about the exterior surface.

3. The filling head according to claim 1, wherein the container comprises a pocket sized to receive at least one of the plurality of raised surfaces of the exterior surface of the receptacle, and the exterior surface of the filling head comprises a protuberance disposed on an edge of the filling head configured for engagement with an indent on the container.

4. The filling head according to claim 3, wherein a plurality of pockets are disposed radially on a disc, each pocket sized to receive at least one of the plurality of raised surfaces of the exterior surface of the receptacle to allow filling of the pocket with the powder.

5. The filling head according to claim 1, wherein the powder comprises a medicament.

6. The filling head according to claim 5, wherein the medicament comprises an excipient.

7. The filling head according to claim 1, wherein the powder comprises a powder for a dry powder inhaler.

8. The filling head according to claim 1, wherein the plurality of raised surfaces have a linear configuration.

9. The filling head according to claim 1, wherein the plurality of raised surfaces have a non-linear or angled configuration.

10. The filling head according to claim 1, wherein each of the plurality of raised surfaces comprises at least three openings to allow passage of the powder from the receptacle to the container.

11. The filling head according to claim 1, wherein a conical-shaped projection is disposed substantially in a center of the interior surface, the conical-shaped projection defining an interior channel.

12. The filling head according to claim 11, wherein the filling head defines a channel disposed substantially in the center of the filling head, the channel being configured for disposal of a fixation element comprising a screw to lock the conical-shaped projection onto the interior surface.

13. The filling head according to claim 11, wherein the exterior surface comprises a first flange and a second flange, the first flange being located opposite the second flange.

14. A filling head for filling a container, the filling head comprising: a receptacle, the receptacle comprising: an interior surface configured to receive a powder and having a plurality of circumferentially arrayed openings disposed therein to allow passage of the powder; and an exterior surface, the exterior surface of the receptacle having a plurality of circumferentially arrayed raised surfaces, each of the plurality of raised surfaces having a plurality of openings aligned with at least one of the plurality of openings of the interior surface to allow passage of the powder from the receptacle to the container.

15. The filling head according to claim 14, wherein the container comprises a pocket sized to receive at least one of the plurality of raised surfaces of the exterior surface of the receptacle, and a plurality of pockets being disposed radially on a disc, each pocket sized to receive at least one of the plurality of raised surfaces of the exterior surface of the receptacle to allow filling of the pocket with the powder.

16. The filling head according to claim 14, wherein (i) the powder comprises a medicament, (ii) the powder comprises a medicament comprising an excipient; or (iii) the powder comprises a powder for a dry powder inhaler.

17. The filling head according to claim 14, wherein (i) the plurality of raised surfaces have a linear configuration; or (ii) the plurality of raised surfaces have a non-linear or angled configuration.

18. The filling head according to claim 14, wherein the interior surface further comprises a conical-shaped projection disposed substantially in a center of the interior surface, and each of the plurality of raised surfaces comprising at least three openings to allow passage of the powder from the receptacle to the container.

19.-22. (canceled)

23. A method of filling a unit dose carrier using a filling head, the method comprising: attaching the filling head to a filling machine, the filling head comprising a receptacle, the receptacle having an interior surface configured to receive a powder and having a plurality of circumferentially arrayed openings disposed therein to allow passage of the powder; and the receptacle having an exterior surface, the exterior surface of the receptacle having a plurality of circumferentially arrayed raised surfaces, each of the plurality of raised surfaces having a plurality of openings aligned with at least one of the plurality of openings of the interior surface; loading the interior surface with the powder; engaging the plurality of circumferentially arrayed perforated shapes with a plurality of pockets disposed in the unit dose carrier; attaching the unit dose carrier to the filling head; administering a plurality of taps to the filling head; and filling each of the plurality of the pockets with at least a first layer of powder.

24. The method according to claim 23, wherein after the interior surface is loaded, the powder flows through the plurality of circumferentially arrayed openings, out of the plurality of openings of the plurality of circumferentially arrayed raised surfaces, and into each of the plurality of pockets of the unit dose carrier.

25. (canceled)
